(19) United States
(12) Patent Application Publication
(10) Pub. No.: US 2025/0197315 A1
(43) Pub. Date: Jun. 19, 2025

(54) GREEN ETHYLENE WATER RECYCLE

(71) Applicant: Lummus Technology LLC, Houston, TX (US)

(72) Inventors: Paul Keusenkothen, Houston, TX (US); Mariana Asahi, São Paulo City (BR); Daniela Molina, Porto Alegre (BR); Adler Moura, Campinas (BR); Martinus Almering, Houston, TX (US); William Scott Neifert, Houston, TX (US)

(73) Assignee: Lummus Technology LLC, Houston, TX (US)

(21) Appl. No.: 18/986,493

(22) Filed: Dec. 18, 2024

Related U.S. Application Data
(60) Provisional application No. 63/611,997, filed on Dec. 19, 2023.

(51) Int. Cl. C07C 1/24; C02F 1/00; C02F 1/04; C02F 1/06; C02F 1/44; C02F 9/00; C02F 101/32; C02F 103/36

(52) U.S. Cl. CPC C07C 1/24; C02F 9/00; C02F 1/004; C02F 1/048; C02F 1/06; C02F 1/44; C02F 2101/322; C02F 2103/36

(57) ABSTRACT

Processes and systems for producing olefins, such as ethylene, includes feeding a steam-alcohol mixture to a dehydration reactor and dehydrating the alcohol to form a reaction effluent comprising an olefin and water. The process includes quenching and separating the reaction effluent to recover a quenched effluent and a gaseous hydrocarbon comprising the olefin, The quenched effluent is separated to form a volatile organic stream and an aqueous organic effluent. Solids, hydrocarbons, or both, may be removed from the quenched effluent, the aqueous organic effluent, or both. The process further includes one or both of: feeding a portion of the aqueous organic effluent as the quench medium, or, vaporizing a portion of the aqueous organic effluent, including water and hydrocarbonaceous compounds, to form a mixed dilution steam stream and a residue liquid, and diluting a vaporized oxygenated hydrocarbon feedstock with the mixed dilution steam stream to form the steam-oxygenated hydrocarbon mixture.

### FIELD OF THE DISCLOSURE

Embodiments of the present disclosure generally relate to processes for the formation of ethylene, propylene, and other olefins by dehydration of oxygenated hydrocarbons, such as ethanol, propanol, butanol and other oxygenated species, including those made by green processes (from renewable resources). Embodiments of the present disclosure further relate to efficient use of the water generated during the dehydration processes.

### BACKGROUND

Processes for production of olefins via the dehydration of alcohols produce a significant quantity of water as a reaction product. Unfortunately, there are a significant amount of reaction byproducts that are generally considered to render the resulting produced water unsuitable for reuse, due to fouling or other issues associated with the byproducts.

### SUMMARY OF THE CLAIMED EMBODIMENTS

In one aspect, embodiments disclosed herein relate to a process for producing ethylene. The process includes feeding a steam-ethanol mixture to a dehydration reactor and dehydrating ethanol, in the dehydration reactor, to form a reaction effluent comprising ethylene and water. The process further includes quenching and separating the reaction effluent with a quench medium comprising water to recover a quenched effluent and a gaseous stream comprising the ethylene, and then separating the quenched effluent to form a volatile organic stream and an aqueous organic effluent. During the process, solids, hydrocarbons, or both, may be removed from the quenched effluent, the aqueous organic effluent, or both. The process further includes one or both of: feeding a portion of the aqueous organic effluent as the quench medium used in the quenching and separating step; or, vaporizing a portion of the aqueous organic effluent, including water and hydrocarbonaceous compounds, to form a mixed dilution steam stream and a residue liquid, and diluting a vaporized ethanol-containing feedstock with the mixed dilution steam stream to form the steam-ethanol mixture.

In another aspect, embodiments disclosed herein relate to a process for producing olefins. The process includes feeding a steam-alcohol mixture to a dehydration reactor and dehydrating the alcohol, in the dehydration reactor, to form a reaction effluent comprising an olefin and water. The process further includes quenching and separating the reaction effluent with a quench medium comprising water to recover a quenched effluent and a gaseous hydrocarbon comprising the olefin, and then separating the quenched effluent to form a volatile organic stream and an aqueous organic effluent. During the process, solids, hydrocarbons, or both, may be removed from the quenched effluent, the aqueous organic effluent, or both. The process further includes one or both of: feeding a portion of the aqueous organic effluent as the quench medium used in the quenching and separating step; or, vaporizing a portion of the aqueous organic effluent, including water and hydrocarbonaceous compounds, to form a mixed dilution steam stream and a residue liquid, and diluting a vaporized oxygenated hydrocarbon feedstock with the mixed dilution steam stream to form the steam-oxygenated hydrocarbon mixture.

In another aspect, embodiments disclosed herein relate to a system for the production of filtered water from green ethylene process reaction water containing oxygenated hydrocarbons, ethanol, light hydrocarbons, and suspended solids. The system includes a flash or distillation stage to remove light hydrocarbons from the green ethylene reaction water to produce a water enriched stream, and a secondary treatment stage comprising a filter, membrane, or coalescer to remove at least some suspended solids or foulants or heavy oxygenates from at least a portion of the water enriched stream to produce an aqueous product stream. The system further includes an internal supply stage that vaporizes the aqueous product stream and transports a resulting vaporized aqueous product stream as a feed to a dehydration reactor. The resulting aqueous product stream is an aqueous steam with a chemical oxygen demand (COD) of 5 to 200 mg/l and a suspended solids content of 5 to 200 mg/l.

In yet another aspect, embodiments disclosed herein relate to a system for the production of green ethylene. The system includes a feed preparation unit for treating and vaporizing an ethanol feedstock to produce a vaporized ethanol. The system also includes a dehydration unit for receiving the vaporized ethanol and a superheated steam mixture, the dehydration unit containing a catalyst for dehydrating the alcohol to form a reaction effluent comprising ethylene, water, and reaction byproducts. Further, the system includes a product purification unit configured to quench and separate the reaction effluent to form a raw ethylene product stream comprising ethylene and a first portion of the reaction byproducts and an aqueous effluent stream comprising water and a remaining portion of the reaction byproducts. The system also includes an internal supply stream production unit configured to receive the aqueous effluent stream and to remove a second portion of the reaction byproducts to produce an aqueous product stream comprising water and remaining reaction byproducts, as well as an internal supply system for vaporizing and feeding, as the superheated steam mixture to the dehydration unit, at least a portion of the aqueous product stream.

Other aspects and advantages will be apparent from the following description and the appended claims.

### BRIEF DESCRIPTION OF DRAWINGS

FIG. 1 illustrates a block flow diagram of systems for producing olefins according to one or more embodiments disclosed herein.

FIG. 2 illustrates a simplified process flow diagram of systems for producing olefins according to one or more embodiments disclosed herein.

FIG. 3 illustrates a simplified process flow diagram of a portion of a system for producing olefins according to one or more embodiments herein.

### DETAILED DESCRIPTION

Embodiments herein relate generally to processes and systems for the production of olefins from alcohols. More specifically, embodiments herein relate to processes and systems for the production of “green” olefins from renewable resources. For embodiments requiring a “green” certification, all steps according to embodiments herein may be proven on bio-based feedstocks or bio-based intermediates, and do not include any fossil-based feedstocks or intermediates.

In general, process configurations according to embodiments herein include four primary unit operations. The first unit is a feed preparation unit. The feed preparation may include various operations including desalting, dilution, heating, and other steps to prepare the feedstock for use.

The second unit is a dehydration unit (alcohol dehydration unit or oxygenate dehydration unit) for producing an olefin. The dehydration unit may be used, for example, for the dehydration of an alcohol feedstock, such as bioethanol or biobutanol, to form an olefin, such as ethylene or butene. Other alcohol and bio-alcohol feedstocks may also be used, among other oxygenated hydrocarbons that may be dehydrated to form an olefin.

The third unit is a product purification unit. The product purification unit includes various unit operations to separate and recover the target olefin(s) from the dehydration reaction effluent. The product purification unit may include various unit operations including a quench system, acid gas capture, byproduct separations, solids removal, and other steps to recover and process the reaction effluent to recover the desired olefin product stream(s).

The fourth unit is an internal supply stream purification unit. The dehydration process produces a significant amount of water, as well as various byproducts. Processes herein utilize the produced water, which may be contaminated with byproducts, as an internal aqueous supply stream for performing various unit operations within one, two, or all of the feed preparation unit, the dehydration unit, and the product purification unit. The internal supply stream purification unit may include various unit operations to process the aqueous streams recovered from the product purification unit so as to provide aqueous or organic streams suitable for internal use within the alcohol-to-olefin processes herein.

Feedstocks useful in embodiments herein may include various alcohols, such as C2-C7 alcohols, including one or more of primary alcohols (such as ethanol), secondary alcohols (such as 2-butanol), and iso-alcohols (such as isopropanol). In some embodiments, the alcohol feed may include two or more C2-C7 alcohols, fed separately or as a mixture. In other embodiments, the alcohol feed may include ethanol or a mixture of ethanol and one or more additional alcohols. Primary alcohols useful in embodiments herein may include ethanol, propanol, butanol, pentanol, hexanol, and heptanol. Secondary alcohols useful in embodiments disclosed herein may include 2-butanol, 2-pentanol, 3-pentanol, 2-hexanol, and 3-hexanol, among other alcohols where the OH group is in the 2-, 3-, or 4-position. Iso-alcohols useful in embodiments may include isopropanol, isobutanol, isopentanol, isohexanol, and isoheptanol.

In various embodiments, the alcohols useful in embodiments disclosed herein may include bio-alcohols, such as bio-derived ethanol, bio-derived propanol or isopropanol, or bio-derived butanols, for example. In some embodiments, the bio-alcohols may be produced via fermentation. In other embodiments, the bio-alcohols may be produced via a process including biomass gasification to syngas followed by a modified Fischer-Tropsch synthesis. Bio-alcohols are a feed material that may be derived from renewable resources, such as corn, corn stalks, corn cobs, lignocellulose, sugarcane, sugar beets, and wheat, among others.

For ease of downstream separations, the feedstock alcohol(s) may produce target olefin(s) having a boiling point less than the boiling point of water.

Production of olefins according to embodiments disclosed herein may be accomplished via the dehydration of the alcohol(s). Catalysts useful in embodiments disclosed herein include activity for the dehydration of the alcohols to form olefins and water. Catalysts useful in embodiments herein may include, for example, resin catalysts, such as sulfuric acid resin or hydrochloric acid resin catalysts. Other catalysts that may be used include various grades of alumina, silica, alumina silicates, or other acidic catalysts that have dehydration activity. Side (byproduct) reactions may include, among others, the formation of light hydrocarbons (i.e., C1-C6 paraffins, including methane, ethane, propane, butanes, etc.), methanol, aldehydes such as propanal or acetaldehyde, acetates such as ethyl acetate, heavier alcohols (ethanol→sec-butyl alcohol, for example), 2-pentanone or other ketones such as acetone, diethyl ether or other dialkyl ethers, higher molecular weight oligomers and ethers, aromatics, and coke, which typically cause fouling of the catalyst.

The alcohol feedstock(s) may be provided as a hydrous or an anhydrous feedstock. Anhydrous as used herein refers to a stream containing less than 0.5 wt % water. Other alcohol feeds useful in embodiments disclosed herein may contain impurities, such as water. For example, alcohols may contain a certain amount of water. Typically, the water is removed from the alcohol. However, as water is a byproduct of the alcohol dehydration reaction, alcohol feeds used in embodiments disclosed herein may include water as an impurity. Excessive water in the feed may decrease reactor conversion equilibrium, and may result in increased reboiler duties, but water as a feed impurity may be tolerated in systems described herein.

In some embodiments, alcohol feeds may include up to 40 weight percent water; up to 30 weight percent water in other embodiments; up to 20 weight percent water in other embodiments; up to 10 weight percent water in other embodiments; up to 5 weight percent water in other embodiments; and up to 2 weight percent water in yet other embodiments. In other embodiments, alcohol feeds may be substantially pure alcohol or alcohol mixtures. In other embodiments, alcohol feedstocks useful in embodiments disclosed herein may contain from 0.1 to 100 wt. % alcohol and from 0 to 99.9 wt. % water. In other embodiments, the alcohol feedstock may contain from 10 to 100 wt. % alcohol; from 25 to 100 wt. % alcohol in other embodiments; and from 50 to 95 wt. % alcohol in yet other embodiments. The amount of water that may be used within the catalytic reaction zones may depend on (1) the reaction equilibrium constant and (2) the strength/activity of the acid catalyst for conversion. For example, as one moves from resin type catalysts to stronger sulfuric or hydrochloric acid concentrations, activity can be maintained at higher water concentrations. Acid resin catalysts will be more susceptible to loss in catalyst activity as one moves to larger quantities of water at elevated temperatures.

The above-described alcohol feedstock(s) are fed to the feed preparation unit. The feed preparation unit may include one, or more, unit operations including desalting, dilution, heating, distillation, reactors, and other steps to remove impurities and to appropriately prepare the feedstock for use. For example, alcohol feedstocks that are dilute or contain a significant amount of water may be processed to remove a portion of the water or another impurity, such as by distillation or membrane separation, so as to provide an alcohol feed suitable for downstream processing.

The alcohol feedstock may be provided as a hydrous mixture. In other embodiments, the alcohol may be provided as an anhydrous feed or with a low (less than 10 wt %) water content. The as-provided alcohol may contain residual salts or material separation agents (such as benzene or other agents where azeotropic distillation is used to purify the alcohol (ethanol) feedstock provided), as well as denaturants or other impurities. In such embodiments, the alcohol feedstock may be processed in the feed preparation unit to remove salts, separation agents, and/or denaturants prior to vaporization and feed of the alcohol to reactors herein. Salt removal may minimize fouling of heat exchangers, heating coils, or reactors/catalysts, and the salt removal may be performed, for example, by passing the alcohol feedstock through a treatment system to remove the salts (anions and cations) contained in the feed. Treatment systems useful herein may include anion exchange beds, cation exchange beds, mixed bed ion exchange, alumina- or iron-based adsorbent beds, lime softening, filtration, reverse osmosis, and other treatment techniques to remove the salts contained in the feed. In some embodiments, the feedstock may be passed serially through an anion exchange bed and a cation exchange bed, in either order. In other embodiments, the feedstock may be diluted with an internally generated dilution water stream prior to salt removal, such as passing the diluted feedstock serially through an anion exchange bed and a cation exchange bed, in either order. Material separation agents such as benzene may also be removed, such as via adsorption, if desired. However, the material separation agents are generally tolerated by the dehydration catalysts and may be addressed via processing downstream of the dehydration reactor. Similarly, hydrocarbon or alcohol denaturants may be tolerated by the dehydration catalysts and may be addressed via processing downstream of the dehydration reactor.

The resulting prepared alcohol feed may then be heated for feed of a vaporized alcohol to the second unit. Vaporization of the alcohol feed may be performed in a convective section of a furnace, a boiler, a heat exchanger, or an electric heater, for example. Indirect or direct heat exchange may be used.

In some embodiments, following vaporization of the alcohol feedstock, the alcohol feedstock may be combined with a superheated steam stream to provide a superheated alcohol-steam stream for feed to the endothermic dehydration reactor. In some embodiments, the superheated steam is a mixed dilution steam stream, described further below, derived from a water steam containing various dehydration reaction byproducts, where the water stream is recovered in the product purification unit and internal supply stream purification unit.

The amount of dilution steam used may depend upon the alcohol feedstock used, as well as the reactor configuration. In general, the amount of steam to alcohol in the dehydration reactor feed is at a mole ratio in a range from 0.5:1 to 1.5:1, such as from 0.7:1 to 1.3:1. In other embodiments, the amount of steam to alcohol in the dehydration reactor feed is at a mass ratio in a range from 2.0:1 to 3.0:1, such as from about 2.4:1 to 2.8:1. On a mass basis, the dehydration reaction may result in a reaction effluent having roughly double the amount of water as compared to the feed. The amount of dilution steam used may depend upon the alcohol feedstock, the dehydration catalyst, dehydration reaction conditions, steam temperature, and other factors. In general, the superheated dilution steam should provide sufficient energy to the system to sustain the catalytic activity in the endothermic dehydration reactor.

Dehydration reactors useful in embodiments disclosed herein may include traditional fixed bed reactors, bubbling bed reactors, motive bed reactors, and other types of dehydration reactors known in the art. Reactors useful in embodiments disclosed herein may be used as a stand-alone reactor or may be used in combination with one or more reactors of the same or different type. Multiple-reactor systems useful in embodiments disclosed herein may include a series of the same type of reactor or reactors in parallel, or different types of reactors in series. A person of ordinary skill in the art would recognize that other types of reactors may also be used.

Reaction conditions in the dehydration reactor may depend upon the alcohol(s) being processed, the catalyst(s) being used, and other factors. For example, reaction temperatures may be in a range of 50° C. to 500° C. and pressures may be in a range from 0.01 to 20 MPa (gauge). In some embodiments, the dehydration reactor temperature may be in a range from a lower limit of 100, 125, 150, 175, 200, 225, 250, 275, 300, 325, or 350° C. to an upper limit of 250, 275, 300, 325, 350, 375, 400, 425, 450, 475, or 500° C., where any lower limit may be combined with any upper limit. In some embodiments, the dehydration reaction pressure may be in a range from a lower limit of 0.1, 0.2, 0.3, 0.5, 1.0, 1.5, or 2.0 MPa (gauge) to an upper limit of 1.0, 1.5, 2.0, 2.5, 3.0, 4.0, or 5.0 MPa (gauge), where any lower limit may be combined with any upper limit.

Following conversion in the dehydration unit, the resulting dehydration reactor effluent may be fed to the product purification unit. In some embodiments, the resulting dehydration reactor effluent may be initially cooled, such as in a feed/effluent exchanger and then fed to the product purification unit.

Product purification units useful in embodiments herein may include a quench or wash tower. The quench tower contacts and cools the vaporous reaction effluent with a quench medium. The quench may be performed at conditions suitable for condensation and capture of water, oxygenated hydrocarbons (unreacted ethanol, oxygenated reaction byproducts), and any heavier hydrocarbons (oligomers, etc.) by the quench medium, thereby producing a raw olefin stream and an aqueous effluent.

The product purification unit may also include one or more distillation columns, extractive distillation columns, or strippers to separate the olefin product from other components contained in the raw olefin stream. For example, the raw olefin stream may be compressed and fed to a first distillation column to separate heavier (higher boiling) components from the raw olefin stream, recovered as a bottoms, and the resulting olefin-containing overheads stream may be fed to a second distillation column or a stripper to remove light (lower boiling) components contained in the raw olefin stream, recovered as an overheads, from the purified olefin, recovered as a bottoms. Driers, compressor and compressor knock-out drums, and other components may be used to remove and/or recover any water carried over in the raw olefin stream.

Quench media useful in embodiments herein include water or aqueous mixtures. In some embodiments, the quench medium may include an alkaline or amine component to absorb acid gases contained in the reaction effluent. In other embodiments, the product purification unit may include a water scrubber (water quench) unit followed by an alkaline wash column. In some embodiments, the water used as a quench medium or in an alkaline wash column may be an aqueous stream derived from the internal supply stream purification unit.

The aqueous stream(s) recovered from the product separation unit may be fed to the internal supply stream purification unit. The internal supply stream purification unit receives the aqueous effluent from the quench tower or water scrubber, and possibly other waters produced during recovery of the olefin product. The aqueous stream(s) may include water and various reaction byproducts, as noted above, including various oxygen-containing hydrocarbons, unreacted alcohol, sodium hydroxide, sodium carbonate, and possibly polymer or oligomer, formed from the olefins or as a byproduct reaction with the alkaline agent, such as oligomerization of aldehydes (acetaldehyde, for example) or ketones (such as acetone) catalyzed by caustic agents.

To process the aqueous stream(s) received, the internal supply stream purification unit may include various solid and hydrocarbon removal units, including filtration, biological filters, oxidative processes, photolysis, distillation columns or strippers, membrane separation units, membrane bioreactors, adsorption beds, coalescers, reverse osmosis units, or other various separation devices or combination of separation devices to recover a water stream of sufficient purity for use in upstream units. The internal supply stream purification unit may produce, for example, a volatile organic stream, comprising volatilizable organic compounds contained in the aqueous stream(s), a mixed water/organic feed stream, and a mixed water/organic discharge stream, where the mixed water/organic streams contain non-volatilizable organic compounds contained in the aqueous streams. In some embodiments, the volatile organic stream may be used as a fuel gas feed stream that may be combusted to generate heat or power for use in one or more of the various unit operations of the processes to produce olefins described herein (boilers/steam generators, heaters/heating coils (radiant or convective), etc.).

In some embodiments, the internal supply stream purification unit includes a solids/hydrocarbons removal unit and an effluent treatment column. The solids/hydrocarbons removal unit may include a coalescer, settling drums, filters, or other unit operations or combination of unit operations suitable to facilitate separation of at least a portion of the solids and at least a portion of the hydrocarbons from the water. The solids/hydrocarbon removal unit may be disposed or located upstream of the effluent treatment column, downstream of the effluent treatment column, or both upstream and downstream of the effluent treatment column.

The effluent treatment column may be a distillation column or stripper that may be used to separate volatilizable organic compounds from water and heavier organic compounds contained in the aqueous stream(s). The volatilizable organic compounds may be recovered as an overheads from the effluent treatment column, and may be used, for example, as a fuel gas feedstock for a furnace, heater, or energy conversion unit within the system or at the plant.

In some embodiments, in addition to the volatile organic overheads stream, an alcohol containing side draw may also be withdrawn from the effluent treatment column. The alcohol containing side draw may be combined with the alcohol feedstock upstream, within, or downstream of the feed preparation unit. If necessary, the alcohol containing side draw may be processed to provide an alcohol rich stream or a purified alcohol stream to be combined with the alcohol feedstock.

In one embodiment, the internal supply stream purification unit includes a solids/hydrocarbon removal unit followed by an effluent treatment column. The aqueous stream recovered as a bottoms from the effluent treatment column may be divided into a discharge stream and an aqueous supply stream.

In other embodiments, the internal supply stream purification unit includes an effluent treatment column followed by a solids/hydrocarbon removal unit. The aqueous stream recovered as a bottoms from the effluent treatment column may be divided into a discharge stream and an aqueous supply stream, where the aqueous supply stream is further treated in the solids/hydrocarbon removal unit.

In yet other embodiments, the internal supply stream purification unit includes a solids/hydrocarbon removal unit both upstream and downstream of the effluent treatment column. The aqueous effluent from the upstream solids/hydrocarbon removal unit may be fed to the effluent treatment column. Subsequently, the aqueous stream recovered as a bottoms from the effluent treatment column may be divided into a discharge stream and an aqueous supply stream, where the aqueous supply stream is further treated in the downstream solids/hydrocarbon removal unit.

The resulting aqueous product stream produced within the internal stream purification unit may be used, as noted above, as an internal supply stream for performing various unit operations within one, two, or all of the feed preparation unit, the dehydration unit, and the product purification unit. In some embodiments, a portion of the aqueous product stream may be used as a diluent to dilute an alcohol feedstock upstream of the feed preparation unit. In some embodiments, a portion of the aqueous product stream may be fed to a steam generation unit to be vaporized, or vaporized and superheated, and used as a mixed (steam plus organics) dilution steam stream combined with the vaporized alcohol feedstock upstream of the dehydration unit. In some embodiments, a portion of the aqueous product stream may be used as a quench medium or as an aqueous feed component for an alkaline wash unit. The volume of the aqueous product stream recovered, as compared to the aqueous discharge stream, may thus depend upon the total expected requirements of the aqueous product stream for the various uses noted above.

Vaporization of the aqueous product stream may be conducted in a boiler, heat exchanger, electric heater, or in a heating coil disposed in a convective or radiant section of a furnace, for example. Superheating of the resulting mixed steam stream may be conducted in similar units.

In some embodiments, due to the possible carryover of solids or other contaminants with the aqueous product stream, the mixed dilution steam stream may be produced in two heating stages, including an initial heating stage to produce a wet (partially vaporized) steam stream. The wet steam stream may then be sprayed or otherwise processed to separate water droplets, which may contain the entrained solids, from steam. The resulting vaporized mixture, at its dew point, may then be superheated to form the superheated mixed dilution steam stream. The blowdown, which may include water, solids, and some organics, may then be cooled and combined with the discharge water for further processing and treatment before disposal of the excess water. In some embodiments, the hot discharge water may be used as a heat exchange medium, such as a feed/effluent exchanger to preheat the aqueous product stream prior to vaporization, to preheat the aqueous effluent from the quench tower upstream of the effluent treatment column, or both, for example.

The bottoms stream recovered from the effluent treatment column includes, as noted above, water, solids, and hydrocarbons. In some embodiments, the bottoms stream recovered from the effluent treatment column may have, for example, a suspended solids content in a range from about 500 to about 5000 mg/L and may have a chemical oxygen demand (COD) in a range from about 500 to about 5000 mg/L. Separation of solids and hydrocarbons from the bottoms stream in the solids/hydrocarbon removal unit may result in an aqueous product stream having a suspended solids content in a range from about 5 to about 200 mg/L and a COD in a range from about 5 to about 200 mg/L. Use of a solids/hydrocarbon removal unit upstream of the effluent treatment column or both upstream and downstream of the effluent treatment column may provide different results (expected higher solids and COD with only upstream removal, and lower solids and COD with both upstream and downstream removal).

Treated water produced by embodiments herein, having an acceptable COD, may be used for various purposes within, or outside, of the system for producing olefins. For example, the treated water may be used as one or more of a cooling water makeup stream, a process water makeup stream, a utility steam makeup stream, a farm irrigation water stream, a wastewater discharge stream, or a potable water stream.

In addition to the collection and use of process generated waters (water resulting from dehydration of alcohols) as a source of feed waters, embodiments herein further contemplate the use of utility discharge waters as a source of feed waters. For example, one or more heat exchangers in the system for producing olefins (such as ethylene) may require a steam heater, such as a distillation column reboiler or a feed preheater, feed heater, or feed superheater. Condensate streams recovered from these various unit operations may be collected and used as a water supply stream, such as a cooling water makeup stream, a process water makeup stream, a utility water makeup stream, a wash water stream, or as a dilution water stream. In some embodiments, the condensate may be mixed and volatilized by a superheated alcohol stream (a superheated ethanol stream) to form a steam-alcohol mixture (a steam-ethanol mixture).

In some embodiments, the condensate or treated water may be used as a diluent water stream mixed with the alcohol feedstock upstream of a salt removal bed. In other embodiments, the condensate or treated water may be used as a process water makeup stream, being fed as one or more of a quench water makeup stream or a wash water makeup stream.

Any excess water generated and not used as a feed water supply or other makeup stream may be discharged from the system as a wastewater discharge stream.

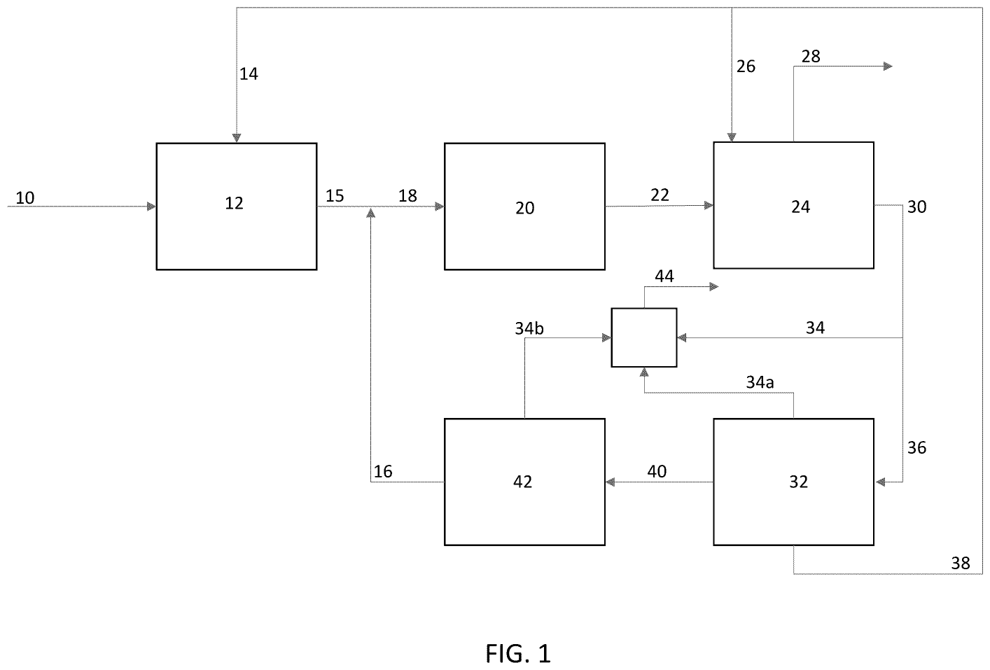

Referring now to FIG. 1, a simplified block flow diagram of processes according to embodiments herein is illustrated. An alcohol feed stream 10, such as a bio-ethanol feed, is fed to a feed preparation unit 12. If necessary, the alcohol feed stream is diluted with aqueous stream 14. While not illustrated, feed preparation unit 12 may include various operations including filtering, salt removal, and vaporization of the alcohol feedstock.

The resulting prepared alcohol feedstock 15 is then mixed with superheated steam 16 to form a mixed alcohol-steam feed stream 18 fed to dehydration unit 20. In dehydration unit 20, the alcohol is contacted with a dehydration catalyst and dehydrated to form water and an olefin, among other reaction byproducts, some of which may be lighter (lower boiling) than the olefin, some of which may be heavier (higher boiling) than the olefin, and many of which may be soluble in water. The resulting reaction effluent 22 is recovered from the dehydration unit and the effluent is cooled and separated in product purification unit 24. An aqueous stream 26 may be provided as the quench medium, the quench process producing a raw olefin stream 28 and an aqueous effluent stream 30. The raw olefin stream may then be further purified to recover the desired olefin(s).

The aqueous effluent stream 30 is then divided into a discharge water stream 34 and an internal water supply stream 36, which is fed to internal supply stream purification unit 32. Internal supply stream purification unit 32 separates at least a portion of the solids and at least a portion of the organic compounds from the water, and produces an aqueous product stream or multiple aqueous product streams for use within the process. As illustrated, a first (liquid) aqueous product stream 38 may be used to supply dilution stream 14 to the feed preparation unit 12 and quench medium 26 to product purification unit 24. A second aqueous product stream 40 may be fed to a steam production unit 42, generating superheated steam 16, used as a heat source and diluent to dehydration unit 20; mixed alcohol-steam feed stream 18 may be further superheated in or before dehydration unit 20. Excess aqueous effluent supplied to internal supply stream purification unit 32 may be recovered as a second discharge stream 34a. The steam generation process may also produce blowdown, recovered as water discharge stream 34b, which may be combined with discharge water streams 34 and 34a and fed collectively as discharge water 44 to a downstream water treatment unit (not illustrated). While three internal aqueous supply streams (14, 26, 40), are illustrated, embodiments herein may use only one or two of such internal streams; for example, it may not be necessary to dilute alcohol feedstock 10.

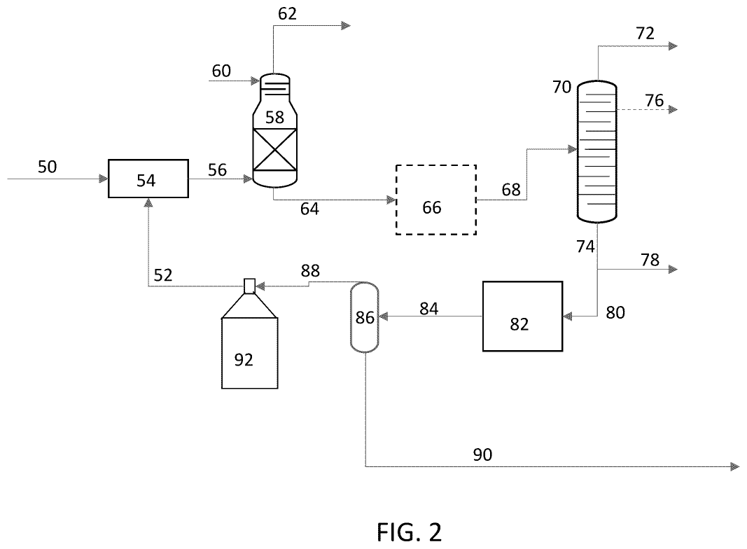

Referring now to FIG. 2, a simplified process flow diagram of systems for producing olefins according to embodiments herein is illustrated. One skilled in the art will appreciate that pumps, valves, heat exchangers, reboilers, overhead condensation systems, and other components used in the process are not illustrated in the simplified process flow diagram. A vaporized alcohol feedstock 50 and a superheated steam stream 52 are fed to a dehydration reactor 54, therein converting the alcohol to an olefin and water; if desired or necessary, the alcohol, steam, or a mixture thereof, may be further superheated prior to introduction into the dehydration reactor 54. The vaporous dehydration reaction effluent 56 is then fed to a quench unit 58, where it is contacted with a quench medium 60 and quenched to separate a bulk of the water (dilution steam and produced water) from the olefin. The raw olefin stream is recovered as an overheads 62 from the quench unit 58, and an aqueous effluent 64 is recovered as a bottoms from quench unit 58. The raw olefin stream 62 may then be further processed (not illustrated) to recover a purified olefin product.

Aqueous effluent 64 is then fed to effluent treatment column 70 for separation of volatilizable hydrocarbons from water. Optionally, aqueous effluent 64 is fed to a solids/hydrocarbon removal unit 66 to remove a portion of any solids and hydrocarbons from the aqueous effluent prior to feed of the resulting partially clarified aqueous effluent 68 to effluent treatment column 70.

In effluent treatment column 70, the aqueous effluent (64, 68) is separated to remove volatilizable hydrocarbons, primarily byproducts of the dehydration reaction, from water. The volatilizable hydrocarbons are recovered as an overheads 72 and the remaining aqueous fraction, including heavier hydrocarbons and possibly some solids, is recovered as a bottoms stream 74. Optionally, an alcohol rich side draw 76 may also be recovered from effluent treatment column 70, and which may be reused as feed to the dehydration reactor 54.

Bottoms stream 74 is then separated into a discharge water stream 78 and an internal water supply stream 80. At least a portion of any remaining solids and hydrocarbons may be removed from internal water supply stream 80, producing an aqueous supply stream 84. Aqueous supply stream 84 is then fed to a dilution steam drum 86, converting at least a portion of the aqueous supply stream to steam 88, and recovering a remaining portion of the aqueous supply stream not vaporized as a discharge stream 90. Steam 88 may then be superheated in furnace 92, producing superheated steam 52 supplied to dehydration reactor 54.

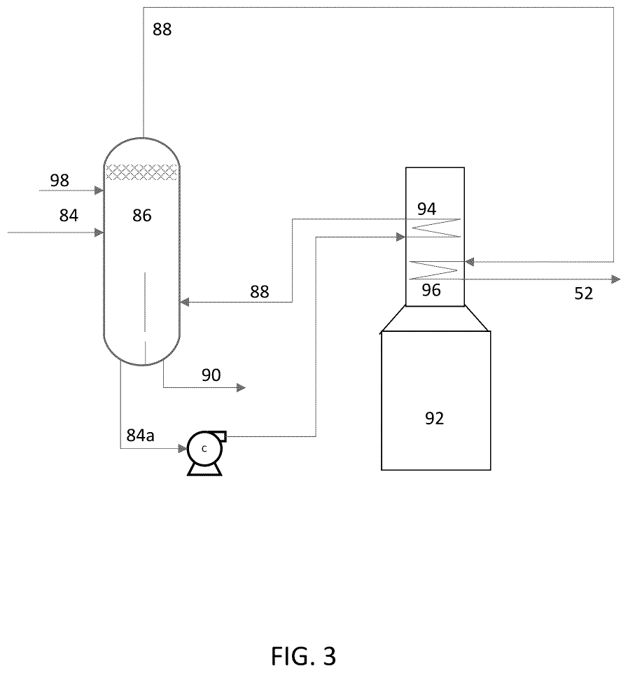

FIG. 3 illustrates one possible process configuration for generating superheated steam according to embodiments herein, where like numerals represent like parts. Aqueous supply stream 84 may be converted to superheated steam in two heating stages. The aqueous supply stream 84 may be fed to a steam drum 86. In an initial heating stage, aqueous effluent 84a is withdrawn from steam drum 86 and pumped, or fed via thermosiphon, through a heating coil 94 within furnace 92 to produce a wet (partially vaporized) steam stream 88a. The wet steam stream 88a may then be sprayed into an internal chamber within steam drum 86 to separate water droplets, which may contain entrained solids, from steam. The resulting steam 88, may then be fed to a second heating coil 96 within furnace 92 to produce a superheated dilution steam stream 52. The blowdown, which may include water, solids, and some organics, may then be recovered from steam drum 86 as discharge stream 90. As also illustrated in FIG. 3, start-up or makeup water or steam may be added to steam drum 86 via flow line 98.

As described above, by removing solids and hydrocarbons from the quenched reaction effluent, embodiments herein allow for water use reduction by internally supplying steam, dilution water, and/or quench water using water generated during the dehydration reaction. The improved filtered vaporized water contaminated with alcohol and other oxygenates further makes an ideal cofeed for diluting anhydrous alcohol feeds, and may also improve ion exchange feed pretreatment. Additionally, the water contaminated with alcohol and other oxygenates can be used for steam generation, with or without hydrocarbon removal.

Unless defined otherwise, all technical and scientific terms used have the same meaning as commonly understood by one of ordinary skill in the art to which these systems, apparatuses, methods, processes and compositions belong.

The singular forms “a,” “an,” and “the” include plural referents, unless the context clearly dictates otherwise.

As used here and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

When the word “approximately” or “about” are used, this term may mean that there can be a variance in value of up to +10%, of up to 5%, of up to 2%, of up to 1%, of up to 0.5%, of up to 0.1%, or up to 0.01%.

Ranges may be expressed as from about one particular value to about another particular value, inclusive. When such a range is expressed, it is to be understood that another embodiment is from the one particular value to the other particular value, along with all particular values and combinations thereof within the range.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

What is claimed as new and desired to be protected by Letters Patent is:

1. A process for producing ethylene, comprising:
feeding a steam-ethanol mixture to a dehydration reactor;
dehydrating ethanol, in the dehydration reactor, to form a reaction effluent comprising ethylene and water;
quenching and separating the reaction effluent with a quench medium comprising water to recover a quenched effluent and a gaseous stream comprising the ethylene;
separating the quenched effluent to form a volatile organic stream and an aqueous organic effluent;
removing solids, hydrocarbons, or both, from the quenched effluent, the aqueous organic effluent, or both; and
one or both of:
feeding a portion of the aqueous organic effluent as the quench medium used in the quenching and separating step; and
vaporizing a portion of the aqueous organic effluent, including water and hydrocarbonaceous compounds, to form a mixed dilution steam stream and a residue liquid, and diluting a vaporized ethanol-containing feedstock with the mixed dilution steam stream to form the steam-ethanol mixture.

2. The process of claim 1, wherein the ethanol is a bio-ethanol and wherein the process is certifiable as a green ethylene process.

3. The process of claim 1, further comprising feeding the residue liquid and a remaining portion of the aqueous organic effluent to a water treatment unit to produce a treated water.

4. The process of claim 3, further comprising using the treated water as one or more of a cooling water makeup stream, a process water makeup stream, a utility steam makeup stream, a farm irrigation water stream, a wastewater discharge stream, or a potable water stream.

5. The process of claim 3, further comprising using the treated water as a process water makeup stream, the process water makeup stream being fed as one or more of a quench water makeup stream, a wash water makeup stream, or a salt removal bed diluent water stream.

6. The process of claim 1, wherein the separating the quenched effluent comprises distilling the quenched effluent to recover a volatile organic stream comprising hydrocarbons, including oxygenated hydrocarbons, and recovering a side draw comprising unreacted ethanol.

7. The process of claim 1, further comprising superheating the mixed dilution steam stream prior to mixing with the vaporized ethanol feedstock.

8. The process of claim 1, further comprising contacting the reaction effluent with an alkaline or amine agent to separate acid gases from the reaction effluent.

9. The process of claim 1, further comprising recovering a condensate stream from one or more heat exchangers used in the process for producing ethylene, and feeding the condensate stream as one or more of a cooling water makeup stream, a process water makeup stream, a utility steam makeup stream, or as a dilution water stream.

10. The process of claim 9, comprising mixing the condensate stream with a superheated ethanol stream.

11. A process for producing olefins, comprising:
feeding a steam-alcohol mixture to a dehydration reactor;
dehydrating the alcohol, in the dehydration reactor, to form a reaction effluent comprising an olefin and water;
quenching and separating the reaction effluent with a quench medium comprising water to recover a quenched effluent and a gaseous hydrocarbon comprising the olefin;
separating the quenched effluent to form a volatile organic stream and an aqueous organic effluent;
removing solids, hydrocarbons, or both, from the quenched effluent, the aqueous organic effluent, or both; and
one or both of:
feeding a portion of the aqueous organic effluent as the quench medium used in the quenching and separating step;
vaporizing a portion of the aqueous organic effluent, including water and hydrocarbonaceous compounds, to form a mixed dilution steam stream and a residue liquid, and diluting a vaporized oxygenated hydrocarbon feedstock with the mixed dilution steam stream to form the steam-oxygenated hydrocarbon mixture.

12. The process of claim 11, wherein the alcohol comprises an alcohol or a mixture of two or more alcohols selected from the group consisting of ethanol, propanol, isopropanol, 1-butanol, 2-butanol, isobutanol, 1-pentanol, 2-pentanol, 3-pentanol, isopentanol, 1-hexanol, 2-hexanol, 3-hexanol, isohexanol, 1-heptanol, 2-heptanol, 3-heptanol, 4-heptanol, and isoheptanol.

13. The process of claim 11, wherein the alcohol comprises ethanol or a mixture of ethanol and one or more alcohols selected from the group consisting of propanol, isopropanol, 1-butanol, 2-butanol, isobutanol, 1-pentanol, 2-pentanol, 3-pentanol, isopentanol, 1-hexanol, 2-hexanol, 3-hexanol, isohexanol, 1-heptanol, 2-heptanol, 3-heptanol, 4-heptanol, and isoheptanol.

14. The process of claim 11, wherein the reaction effluent comprises the olefin, water, and one or more reaction byproducts selected from the group consisting of C1-C8 paraffins, ketones, aldehydes, ethers, methanol, alcohols, acetates, oligomers of the olefin, and aromatics.

15. The process of claim 11, wherein the quenched effluent comprises water, light hydrocarbons, unreacted alcohol, dissolved oxygenated hydrocarbon byproducts, and suspended solids.

16. The process of claim 11, wherein the gaseous hydrocarbon comprises the olefin and at least a portion of hydrocarbonaceous byproducts produced in the dehydration reactor.

17. The process of claim 16, further comprising separating the olefin from the hydrocarbonaceous byproducts in the gaseous hydrocarbon to recover a purified olefin product stream.

18. The process of claim 11, wherein the volatile organic stream comprises one or more of light hydrocarbons and oxygenated hydrocarbons.

19. The process of claim 11, wherein the aqueous organic effluent comprises water, dissolved hydrocarbons, and suspended solids.

20. The process of claim 11, further comprising feeding the residue liquid and a remaining portion of the aqueous organic effluent to a water treatment unit.

21. The process of claim 11, further comprising superheating the mixed dilution steam stream prior to mixing with the vaporized oxygenated hydrocarbon feedstock.

22. The process of claim 11, further comprising contacting the reaction effluent with an alkaline or amine agent to adsorb acid gases from the reaction effluent.

23. The process of claim 11, wherein the aqueous organic effluent has a suspended solids content in a range from about 500 to about 5000 mg/L and a chemical oxygen demand (COD) in a range from about 500 to about 5000 mg/L, and wherein, following the step removing solids and hydrocarbons from the aqueous organic effluent, the resulting aqueous product stream has a suspended solids content in a range from about 5 to about 200 mg/L and a COD in a range from about 5 to about 200 mg/L.

24. A system for the production of filtered water from green ethylene process reaction water containing oxygenated hydrocarbons, ethanol, light hydrocarbons, and suspended solids, comprising:
a flash or distillation stage to remove light hydrocarbons from the green ethylene reaction water to produce a water enriched stream;
a secondary treatment stage comprising a filter, membrane, or coalescer to remove at least some suspended solids or foulants or heavy oxygenates from at least a portion of the water enriched stream to produce an aqueous product stream;
an internal supply stage that vaporizes the aqueous product stream and transports a resulting vaporized aqueous product stream as a feed to a dehydration reactor;
where in the aqueous product stream is an aqueous steam with a chemical oxygen demand (COD) of 5 to 200 mg/l and a suspended solids content of 5 to 200 mg/l.

25. A system for the production of green ethylene, the system comprising:
a feed preparation unit for treating and vaporizing an ethanol feedstock to produce a vaporized ethanol;
a dehydration unit for receiving the vaporized ethanol and a superheated steam mixture, and containing a catalyst for dehydrating the alcohol to form a reaction effluent comprising ethylene, water, and reaction byproducts;
a product purification unit configured to quench and separate the reaction effluent to form a raw ethylene product stream comprising ethylene and a first portion of the reaction byproducts and an aqueous effluent stream comprising water and a remaining portion of the reaction byproducts;
an internal supply stream production unit configured to receive the aqueous effluent stream and to remove a second portion of the reaction byproducts to produce an aqueous product stream comprising water and remaining reaction byproducts;
an internal supply system for vaporizing and feeding, as the superheated steam mixture to the dehydration unit, at least a portion of the aqueous product stream.

26. The system of claim 25, wherein the internal supply system further comprises a feed line for feeding at least a portion of the aqueous product stream to the product purification unit.

27. The system of claim 25, wherein the internal supply system further comprises a feed line for feeding at least a portion of the aqueous product stream to the feed preparation unit.
